(19) United States
(12) Patent Application Publication
(10) Pub. No.: US 2009/0171317 A1
(43) Pub. Date: Jul. 2, 2009

(54) Self-Catheterization Device To Administes Compounds To The Bladder

(76) Inventor: Ebrahim Versi, Gladstone, NJ (US)

Correspondence Address: W SCOTT MCNEES, P.O. BOX 124, PENNINGTON, NJ, 08534, US

(21) Appl. No.: 12/225,084

(22) PCT Filed: Mar. 10, 2006
(86) PCT No.: PCT/US2007/005785

§ 371 Date: Sep. 10, 2008

Related U.S. Application Data
(60) Provisional application No. 60/781,244, filed on Mar. 10, 2006.

(51) Int. Cl. A61M 25/14; A61K 39/08; A61P 13/10

(52) U.S. Cl. 604/517; 604/246; 424/236.1

(57) ABSTRACT

Devices and methods for self catheterization and for instilling fluid into the bladder are disclosed. A catheter device is provided for inserting into the urethra of an individual by the subject him or herself for the purpose of instilling a therapeutic compound into the bladder. The catheter assembly includes a catheter that has an opening near the tip, a valve mechanism, and a reservoir at the opposite end from the tip. The catheter may be provided with separate channels for draining the bladder and instilling a therapeutic compound into the bladder. Methods for self catheterization and self-administration of a therapeutic compound into the bladder by a patient are disclosed.

This application is a national phase application of PCT/US07/005785 and claims priority of U.S. Application No. 60/781,244, filed Mar. 10, 2006; U.S. Application No. 60/790,730, filed Apr. 10, 2006; and U.S. Application No. 60/802,069, filed May 19, 2006.

### FIELD OF THE INVENTION

The present invention relates to devices and methods for a patient to self catheterization and self-administer therapeutic agents into the urinary bladder.

### BACKGROUND OF THE INVENTION

Bladder disease afflicts a large and diverse patient population and includes infectious, functional and malignant disorders. Infectious disorders of the bladder are usually caused by a bacterium. Most of the acute infections can be adequately treated with antibiotics but recalcitrant cases could be treated by intravesical instillation of antibiotic. Further, in patients who have recurrent urinary tract infection, the cause may be a deficiency of the defense mechanism such as an impaired barrier to infection in the lining of lumen of the bladder (e.g., glycosaminoglycans (GAG) layer) or an immunological deficiency. In these cases, instillation of a therapeutic compound into the bladder would be beneficial. Examples of functional diseases are urge incontinence or neurogenic incontinence, unstable bladder, detrusor overactivity, overactive bladder, frequency urgency syndrome and interstitial cystitis. Malignant disorders of the bladder include carcinoma in situ, transitional cell carcinoma, squamous cell carcinoma and adenocarcinoma. The bladder is the most common site of cancer in the urinary tract. Initial treatment is often with local excision and fulguration but follow up treatment with anti-cancer drugs instilled into the bladder would become more widespread if it was practical.

Medical treatment of these disorders has traditionally been by systemic therapy. This results in side effects due to action on other body systems and in many cases not enough of the active compound gets to the lumen of the bladder where its effect would be more pronounced. Intravesical instillation of therapeutic compounds in many cases is a better approach because the therapeutic agent is delivered locally to the target tissue and also because higher doses can be used as systemic side effects are avoided or minimized in cases where there is absorption from the lumen of the bladder. Also this route of administration allows the use of therapeutic compounds that might be toxic if given systemically. In addition this method of delivery can result in adequate concentrations getting to the lumen of the bladder which may not be achieved by systemic administration with excretion via the renal system.

The problem with intravesical therapy is that it is impractical in terms of healthcare policy. Many of these therapies require repeated administration and in some cases the administration would have to be daily or more frequently. Also in some cases such as compounds used to augment the GAG layer, it may be desirable for the bladder instillate to be in contact with the urothelium for a protracted time. This could be achieved by instillation last thing at night. Such treatments would only be possible for in-patients. Because of the expense of in-patient care, treatment protocols have not been set up and research studies have not been done to develop treatment regimens for many potentially beneficial intravesical therapies.

Some patients (male and female) who are unable to voluntarily void (empty the bladder) have been taught the technique of self-catheterization to empty the bladder. In the past the treatment was with an indwelling urinary catheter with all its complications such as infection, encrustation and even erosion. Clean intermittent self catheterization (CISC) as a treatment for these patients has revolutionized therapy as many of these patients no longer require prolonged indwelling catheters. Paradoxically these patients using CISC actually have a lower urinary tract infection rate despite the theoretical risk of the catheter introducing infection. One reason may be that stagnant urine does not remain in the bladder to act as a reservoir for culture of bacteria that could cause infection.

Treatment protocols involving self catheterization and self-instillation of therapeutic agents into the bladder have not been developed because physicians have thought that patients would not be able to carry out such procedures. Further there is a concern that patients who are not medically trained are more likely to introduce infection into the bladder during the catheterization procedure. Also, because for many conditions where such an option may be viable (see above), the patients would find the procedure uncomfortable or painful. All these objections can be overcome. Patients can be taught to catheterize themselves as they have for bladder emptying and the additional act of instilling a therapeutic compound into the bladder should not be difficult to teach. The use of a local anesthetic such as lidocaine to insert the catheter would mitigate the pain or discomfort. The addition of an antibiotic or anti-microbial to the instillation medium would reduce the probability of any infective organism introduced by the catheterization procedure from proliferating and causing an infection.

Whereas CISC and intravesical instillation of therapeutic agents by care givers have been suggested, self-administered therapy by instillation by the patient has not been advocated. The reason for this is that within the medical paradigm, such a logical leap is not obvious and physicians are reluctant to advocate such therapy. However by introducing self administration, many different untried therapies will become possible with consequent improvement in patient care.

In dexterous individuals it has become relatively commonplace for intermittent catheterization of an individual's urinary bladder to be employed, as opposed to placement and maintenance of an indwelling catheter that continuously drains urine from the bladder. This can be done in a hospital setting, a nursing home, doctor's office, rehabilitation facility or, more commonly, in the home. For the latter, patients are often trained to catheterize themselves, a procedure called intermittent self-catheterization. This is usually done to treat such conditions as urinary retention, the inability to evacuate urine, but can also be employed to produce a sample of urine.

There arise many clinical situations where it is necessary to instill a therapeutic agent directly into the bladder. An example of this might be the instillation of a local anesthetic into the bladder to treat bladder pain or a chemotherapeutic agent to treat bladder cancer. This is done by a healthcare provider in a healthcare setting. Given the inconvenience to patients, such a therapeutic modality is not popular or feasible for long term repeated therapies. Also, in some cases, the ideal application would necessitate the instilled compound to remain in contact with the lining of the bladder for as long as possible before the bladder is emptied. The best time to perform such a procedure would be last thing prior to sleep. For such a regimen, the patient would need to self-administer the treatment in the home.

Intermittent self-catheterization followed by bladder instillation would be a solution for the above difficulties and the present invention will allow patients to self-administer. In this way various new therapies that were hitherto impractical will become possible and even desirable. However for this to occur, a device needs to be available which allows both self catheterization and self medication. The present invention is such a device and it allows physicians to easily train patients to perform self catheterization and bladder instillation of a therapeutic agent in the privacy of their own home or other suitable toilet facility at anytime of the day or night.

Some bladder disorders will be particularly amenable to instillation of therapeutic agents by self-catheterization because this procedure permits convenient administration of the drug at any time. Some therapies are most effective when administered when they can remain in the bladder for the longest period of time before being eliminated by voiding of the bladder.

For example, cystitis is inflammation of the bladder and can have many different causes such as infection, radiation, malignancy and in many cases the cause is unknown or poorly understood as in the case of interstitial cystitis.

Symptomatically, cystitis is characterized by bladder pain, increased urinary urgency, increased voiding frequency and increased nocturia. Its duration can be transient or long lasting. However its course presents, it does result in a significant impairment of the quality of the patient's life.

It is hypothesized that a common cause or outcome of cystitis is disruption of the glycosaminoglycan (GAG) layer, which lines the inner surface of the urinary bladder. This GAG layer consists of mucopolysaccharides attached to a core protein that, in turn, is bound to a central hyaluronic acid string. This highly viscous, highly hydrophilic GAG layer protects the bladder epithelium against irritants in the urine including, but not limited to, microorganisms, pathogens, microcrystals, proteins, calcium, urea and carcinogens (Nickel et al. 1993. Journal of Urology, 149:716). When this protective barrier is damaged, the bladder epithelium becomes permeable to urinary irritants, resulting in symptoms of bladder pain, increased urinary urgency, increased voiding frequency and increased nocturia.

Treatment of this GAG layer deficiency can be treated by exogenous administration of hyaluronic acid (HA) but also other compounds such heparin, pentosan polysulfate and chondroitin sulphate. This treatment can be divided into the initial induction phase where the initial repair, replacement and augmentation takes place and then the maintenance phase when the integrity of the GAG layer needs to be maintained. Thus the induction phase regimen would require frequent administration to allow rapid build up of the GAG layer for maximum efficacy of therapy. The frequency of administration during the maintenance phase could be reduced and would be dictated by the need to replace the GAG layer based on its rate of degradation. The ideal administration would be such that voiding of the bladder is delayed as long as possible to allow the exogenous compound, for example HA, to be in contact with the inner lining of the bladder for as long as possible to allow adequate penetration of the HA into the GAG layer and possibly deeper for maximal effect.

To date the treatment regimens of HA advocated have involved the induction phase to consist of weekly administrations followed by monthly administrations for the maintenance phase within a healthcare setting. It is possible that the sub-optimal efficacy of HA in the treatment of interstitial cystitis is a result of this infrequent administration regimen and because the HA is not held within the bladder for a long time. Patients with interstitial cystitis tend to void more frequently and diurnal frequency is greater than nocturnal frequency. These regimens requiring infrequent administration have been advocated based on expediency of needing the treatment to be carried out in a healthcare facility as detailed above.

The advent of self-administration by patients by self-catheterization allows administrations of these compounds such as HA to be carried out in the home. This will not only reduce the cost of such healthcare but the efficacy of HA and other GAG replacement or augmenting compounds should also be increased because more frequent administrations will become feasible. In addition patients will be able to administer such compounds just prior to sleep, allowing the longest possible time for these compounds to stay in the bladder to infiltrate and supplement the GAG layer prior to the next bladder void. The use of GAG layer augmentation is presented as an example. Similar principles would apply for treatment of malignancies of the bladder and other disorders. The devices and methods of the present invention provide similar benefits in administration of any therapeutic agent to bladder.

### SUMMARY OF THE INVENTION

The present invention provides devices and methods for treating bladder disease by instilling a therapeutic agent directly into the bladder. The treatment is self-administered by the patient by clean intermittent self-catheterization. To prevent a urinary tract infection an antibiotic or antimicrobial agent may be added or mixed with the compound to be instilled. To reduce pain related to the procedure, a local anesthetic such as lidocaine may be added or mixed with the compound(s) to be instilled.

The present invention provides a novel catheter assembly that permits patients to perform self-catheterization for the purpose of instilling into the bladder a single or combination of therapeutic agents. The patient empties his or her bladder in the normal way and then, in a sterile manner, inserts the present device into the bladder and introduces the therapeutic agent contained in a reservoir into the bladder. This can be done with the patient in any position be it erect, supine or semi-supine. In situations where the patient is unable to empty the bladder spontaneously, an embodiment of the invention allows the urine to be drained by the catheter and then the passage closed off prior to the instillation of the therapeutic agent.

Certain preferred embodiments of the catheter assembly have the reservoir as a bulb, integral to the device and pre-filled with the therapeutic agent(s) such that the bulb can be squeezed to instill its contents into the bladder. Another embodiment would have the reservoir separate and attachable to the catheter prior to insertion into the bladder. Yet another embodiment would be used in patients who are unable to voluntarily void. In such an instance, the catheter is inserted and the bladder emptied and then a closure mechanism closed off prior to instillation of the reservoir contents into the bladder.

A preferred embodiment of the present invention is a disposable hydrophilic catheter assembly for inserting a catheter into the urethra of an individual for the purpose of instilling a therapeutic compound(s) into the bladder. The catheter may be stiff, semi-flexible or totally flexible and may include a reservoir pre-filled with the therapeutic solution. The catheter may also have a grip-enhanced outer surface for the portion that does not enter the urethra, if desired. The catheter is of a suitable length for use by a male or a female patient and has appropriate internal and external diameters. The catheter has a portion that enters the urethra which is smooth surfaced with an opening or aperture usually off-set at the tip. The other end has the reservoir attached and within the lumen of the catheter is a valve to prevent back flow of the therapeutic solution back into the reservoir.

A membrane or diaphragm may be situated at the opening of the reservoir internally to prevent premature passage of the therapeutic solution into the catheter. This diaphragm or seal is ruptured when the reservoir is squeezed or pressure applied. The membrane holds the therapeutic solution inside the reservoir until instillation is required. The membrane also helps to seal the reservoir connection such that the joint is resistant to infiltration by outside contaminates and prevents backflow into the reservoir. In this way the contents of the reservoir are maintained in a sterile environment.

The present invention also provides a method for collecting a urine sample where the catheter that is used empties the bladder into a collection vessel that can be attached to the end of the catheter out of which urine flows.

A device and method is provided for preventing, improving and/or treating cystitis of any type and prevention of recurrent urinary tract infections in humans, comprising administering into the bladder compounds that have the effect of improving the function of the glycosaminoglycans (GAG) layer of the inner lining of the bladder. Such compounds may replace, supplement, replenish, repair or in some such way augment the GAG layer. Such compounds comprise but are not limited to compositions such as hyaluronic acid (HA), heparin, pentosan polysulfate and/or chondroitin sulphate along with a pharmaceutically acceptable carrier. Administration of these compounds in appropriate amounts would be used to effectively prevent, reduce and/or treat the different forms of cystitis or prevent recurrent urinary tract infections. The method involves self-administration of the compound by the patient into the bladder by transurethral self-catheterization. As this is self-administered, it can be done in the patient's home and can be done numerous times in a day. A particular advantage of this method is that compounds can be administered just prior to sleep to allow the longest possible time for the compound to be in contact with the inner lining of the bladder prior to bladder voiding and hence elimination of the compound which is not adherent to the bladder lining or wall or has not been absorbed. This increased contact time would maximize the quantity of the compound that is absorbed or adsorbed.

### BRIEF DESCRIPTION OF THE DRAWINGS

FIG. 1 is a cross section view of a catheter assembly for instilling a therapeutic agent into the bladder.

FIG. 2 is a cross sectional view of a catheter assembly for voiding the bladder and instilling a therapeutic agent into the bladder.

### DETAILED DESCRIPTION OF THE INVENTION

The present invention provides a disposable or reusable self-catheterization device for inserting into the urethra of an individual for the purpose of instilling a therapeutic compound into the bladder. The invention permits self-administration of a fluid into the bladder by the patient, or administration by another person who does not need to be a health care professional or specialist. The catheter assembly includes a catheter which can be rigid or semi-rigid and has an opening at the tip or offset from the tip, a valve mechanism in the stem of the catheter and a reservoir at the opposite end from the tip. The reservoir can be detachable or an integral part of the catheter assembly. The valve mechanism ensures that the direction of flow is only from the reservoir through to the opening at or near the tip and not in the opposite direction. An enhancement of the basic design incorporates a further and separate passage within the stem of the catheter from the opening at or near the tip towards the reservoir end of the catheter for the purposes of emptying the bladder much as a standard catheter would function except that this includes a closure mechanism. The length of the stem would be longer for the male patient than for the female patient. A catheterization assembly in accordance with the present invention is suitable for use for self-catheterization or catheterization by a healthcare provider for the purposes of instilling a therapeutic compound into the bladder.

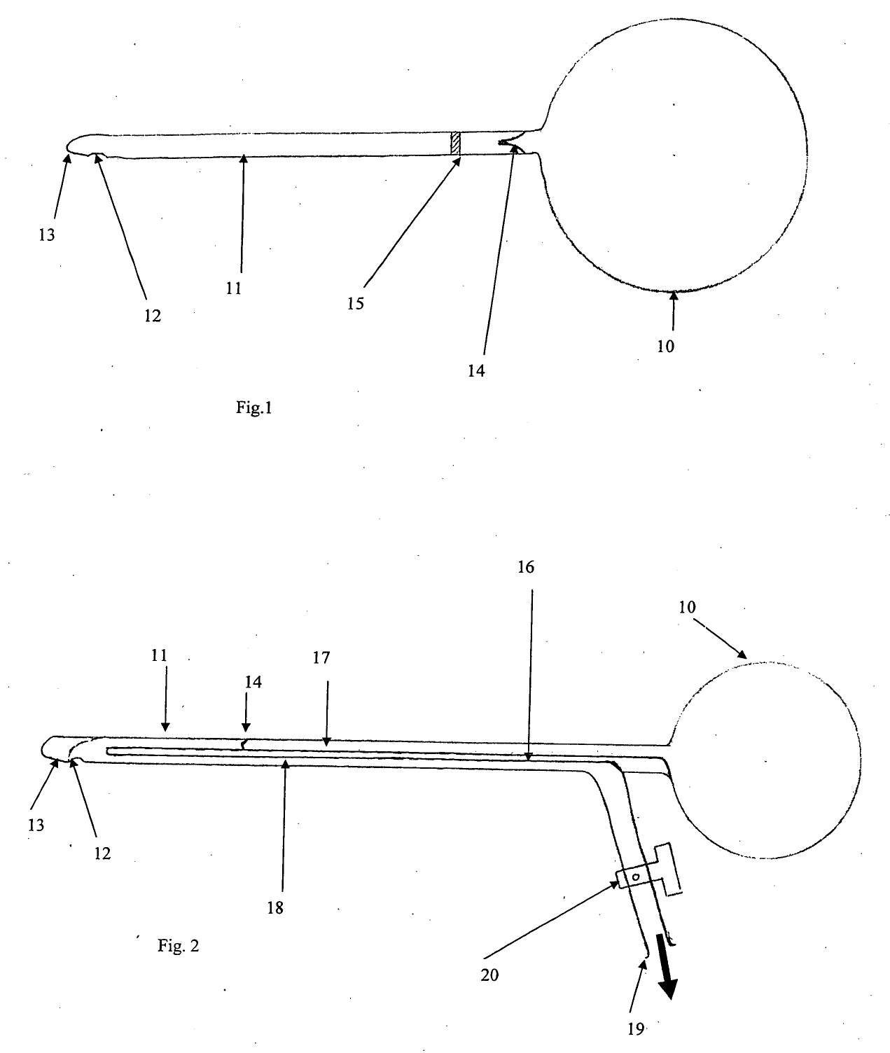

FIG. 1 shows one embodiment of the invention which is used for instilling a therapeutic agent into the bladder. This device is a single unit in which a reservoir (10), which may be pre-filled with the therapeutic solution, is connected to the catheter (11). An aperture (12) is located near the end of the catheter (11), and preferably offset from the tip (13) of the catheter (11). A membrane (not shown) between the reservoir and the valve mechanism keeps the contents of the reservoir from being expelled prematurely. An optional valve mechanism (14) prevents back flow. An optional mark (15) on the outside of the catheter is not essential but can act as a guide to show the patient how far to insert the catheter. The therapeutic solution leaves the catheter into the bladder through the aperture or opening at the tip of the catheter.

FIG. 2 shows another embodiment of the invention which is used for voiding the bladder prior to instilling a therapeutic agent into the bladder. This type of catheter is used by a patient who is unable to void voluntarily to completion. In this embodiment, the lumen of the catheter is divided by a partition (16) along its length into two channels. The first channel (17) connects the reservoir to the distal portion the catheter, near the aperture (12). Therapeutic agent from the reservoir travels through this first channel to the aperture (12) near the end of the catheter (11). The second channel (18) connects the aperture (12) to an outlet (19) near the opposite end of the catheter (11). The first channel (17) and the second channel (18) are in fluid communication near the aperture (12) such that the aperture (12) communicates with both channels. A closure means (20) is disposed in the outlet (19) to open or close the outlet (19) as needed. The catheter is initially inserted into the bladder with the closure mechanism (19) open and the bladder is drained. The closure mechanism (20) is then closed and the reservoir is then emptied into the bladder. A one-way valve means (14) may optionally be disposed within the first channel (17) to prevent backflow of urine toward the reservoir (10) while the bladder is being emptied.

The contents of the reservoir may be expelled from the reservoir by any convenient means. For example, the reservoir may be made of a collapsible material that may be squeezed by hand or compressed by mechanical means, such as a plunger. Alternatively, the reservoir may be a syringe or other similar device.

The procedure of intermittent self-catheterization may increase the probability of contracting a urinary tract infection even though the probability is thought to be lower than when an in-dwelling catheter is left in situ. Thus an embodiment of the present invention could be employed by patients practicing regular clean intermittent self-catheterization. Rather than using oral prophylactic antibiotics which can result in gastrointestinal side effects and the encouragement of resistant bacterial strains, after the bladder is emptied normally or with a device like that shown in FIG. 2, a dose or even a low dose of antibiotic or antimicrobial carried in the reservoir could be instilled directly into the bladder. In this way the therapeutic agent is left in the bladder to prevent any infection from bacteria that might have been introduced by the passage of the catheter.

The present invention thus provides a device and method for treating bladder disease in a subject, comprising self-administration by trans-urethral catheterization into the bladder by said subject of an effective amount of a composition that comprises a chemical compound for the treatment of the bladder disease in combination with an antibiotic or antimicrobial agent for prophylaxis against infection introduced by the catheterization process.

The present invention provides a method for treating bladder disease in a subject, comprising self-administration by trans-urethral catheterization into the bladder by said subject of an effective amount of a composition of a compound that would improve the function of the glycosaminoglycans (GAG) layer on the transitional epithelium of the inner lining of the bladder. Compounds that comprise this category include but are not limited to hyaluronic acid (HA), heparin, pentosan polysulfate and/or chondroitin sulphate along with a pharmaceutically acceptable carrier. The goal of improving the function of the GAG layer is to help in the prevention, improvement and/or treatment of all types of cystitis including that caused by repeated infections. The treatment would consist of the induction phase during which administration is frequent until the GAG layer has been adequately built up. Following this there is the maintenance phase during which administration is less frequent, the goal being to maintain the repaired or replaced GAG layer.

Prior to installation of the therapeutic solution, the patient voids normally to empty the bladder of urine. The patient cleanses the urethral meatus with an antiseptic and then in an aseptic manner the compound is self-administered into the bladder using a self-catheterization device of the present invention. However, if the patient is not able to normally empty his or her bladder, or if it is otherwise desirable, then the patient can self-catheterize to empty the bladder of urine prior to self-administering the compound, possibly though the same catheter used to empty the bladder (FIG. 2). The whole procedure is carried out by the patient in an aseptic manner using a sterile technique.

After the compound is instilled into the bladder, the catheter is removed and the patient tries to avoid emptying his or her bladder for as long as possible. If the compound is to be instilled only once in 24 hours, the procedure is ideally performed just prior to sleep. This will ensure that the instilled compound is exposed to the inner lining of the bladder for the longest time possible.

Where the treatment regimen requires an induction and a maintenance phase, during the induction phase it might be desirable for such instillations to take place even more frequently than every 24 hours. During the maintenance phase the dosing could be once a month or even less often. It is envisaged that treatment regimens will have to be individualized based on the each patient's situation.

A tray made of plastic or similar material specifically designed to hold the items of the catheter kit may be packed with the catheter, lubricant or anesthetic jelly, pre-filled reservoir and optionally one or more antiseptic-soaked swabs, surgical gloves, a specimen container and/or a urine measuring container. The whole kit may be sealed and sterilized in accordance with standard practices for similar medical devices, such as conventional catheterization techniques. The catheter assembly is then stored in a sterile disposable wrapper until needed.

Catheterization may be performed as follows. The patient empties his or her bladder and then the hands are washed thoroughly and the patient then assumes the most comfortable and practical position. The catheter kit is opened and placed flat on a nearby stable surface and the procedure carried with due caution and adherence to aseptic technique. The lubricant or anesthetic jelly is squeezed out of its container on to the tray. The antiseptic-soaked swabs are removed from their wrapping. The urethral area is cleansed with a single wipe of an antiseptic-soaked swab and then care is taken not to contaminate this area before the catheter is inserted. The catheter is picked up by the portion to be held (right of mark in FIG. 1) or held through a plastic sheath covering to maintain sterility of the portion of the catheter that is inserted into the urethra. Care is taken to avoid any contact resulting in contamination of the portion of the catheter to be inserted into to the urethra (left of mark in FIG. 1). The catheter tip is then dipped into the sterile lubricant or anesthetic jelly and is inserted carefully through the external urethral meatus and gently into the urethra up to the mark or until resistance is encountered or pain is experienced.

In the case where the patient is unable to completely empty his or her bladder another embodiment of the device is used (FIG. 2). In this situation the urethral meatus is cleansed and the catheter is inserted, as described above, until urine is seen to flow out. This urine can be collected if desired into a measuring device, a portable waste receptacle, or a specimen container or it can be discarded into the toilet. The ideal scenario for when urine collection is not required is for the urine to pour directly into the toilet. One particular feature of the catheterization assembly of the present invention is that it may be conveniently attached to a collection device with precise measuring marks, to permit more accurate measurement of urine output than is typically possible with conventional disposable catheterization assemblies. This is useful in situations where the therapy is aimed at reducing residual urine volumes and as such the measurements can be made prior to therapy. After the bladder is emptied, the urine outflow passage is closed off and then the reservoir emptied into the bladder.

It is important to note that the user's hands, while preferably being clean, do not have to be strictly sterile nor are sterile gloves essential for this procedure, as long as care is taken not to directly touch the area of the urethral opening or the portion of the catheter that is inserted into the urethra during the catheterization.

Once the contents of the reservoir have been instilled into the bladder, the catheter is removed. The internal valve mechanism is not essential for the functioning of the device for example if a plunger technique were utilized for instilling the therapeutic solution. Even if the balloon reservoir was used without the valve, no backflow of the therapeutic solution would occur if the catheter was removed while the balloon was maintained in its collapsed state. After withdrawal, the catheter is replaced in the tray for easy disposal of the unit in a sanitary manner. The entire catheterization and instillation process would usually be accomplished by a trained patient in less than ten minutes even if the bladder required initial drainage.

The procedure described above enables the user to easily maintain sterile technique to avoid introduction of microbial contaminants into the urethra and bladder. The catheter assembly exemplified herein can be manufactured economically using well known techniques and the therapeutic solution can be pre-filled or in another embodiment could be attached to the catheter.

There are many medical conditions for which this technique can be usefully employed to treat various disorders, including but not limited to bladder inflammation, infection, pain, dysfunction, and cancer. The following examples of therapies are not meant to be an exhaustive list but are included as examples of the types of disorders that may be treated and therapeutic agents that may be administered using the present invention.

Examples of therapeutic compounds which may be instilled into the bladder include, without limitation, those used for the treatment of malignancies, infections and functional disorders of the lower urinary tract. Functional disorders include storage and emptying dysfunctions as well as sensory disorders such as painful bladder. Also this mode of administration could be utilized for preparations aimed at longer duration of drug action and compounds used to enhance uptake through the bladder urothelium.

Various cancers include transitional cell carcinoma, squamous cell carcinoma or adenocarcinoma or some other variety. The cancer could be associated with a condition selected from the group consisting of bladder hyperactivity, irritation, inflammation, micturition pattern alteration, and incontinence. Exemplary therapeutic agents according to the invention include, but are not limited to, chemotherapeutic agents, cytokines, synthetic small molecule drugs, natural products, radionuclides and polypeptides (e.g., proteins). Exemplary chemotherapeutic agents include, but are not limited to, taxane (docetaxel), doxorubicin, mitomycin C, valrubicin, epirubicin, thiotepa, interferon alpha and other cytokines therapeutic activities. The anticancer agent could be selected from the group consisting of doxorubicin, hypochlorous acid, mitoxantrone, camptothecin, cisplatin, bleomycin, cyclophosphamide, methotrexate, streptozotocin, actinomycin D, vincristine, vinblastine, cystine arabinoside, anthracyclines, alkylative agents, platinum compounds, antimetabolites, nucleoside analogs, methotrexate, purine and pyrimidine analogs, adriamycin, daunomycin, mitomycin, epirubicin, 5-FU, and aclacinomycin.

Other exemplary therapeutic agents for the treatment of bladder or other disorders include, but are not limited to, antisense Bcl-2 oligonucleotide; EGF-dextran-Tc (radionuclide to EGF-receptor); BCG; folic acid analogs; methotrexate (MTX); pyrimidine analogs; fluorouracil (5-FU); fluorodeoxyuridine; Cytarabine; purine analogs such as 6-mercaptopurine (6-MP) and 6-thioguanine (6-TG); alkylating agents such as nitrogen mustards, mechlorethamine, cyclophosphamide (Cytoxan.®.), Melphalan and Chlorambucil; natural products, such as vinca alkaloids, vincristine (Oncovin.®.), vinblastine (Velban.®.), vinorelbine (Navelbine.®.), epipodophylotoxins, etoposide (VePesid.®., VP-16), taxol (Paclitaxel.®.), antitumor antibiotics, anthracyclines including doxorubicin hydrochloride (Adriamycin.®.), daunorubicin, idarubicin, mitoxantrone (an Anthracenedione that lacks a sugar moiety), Bleomycin (Blenoxane.®.), Dactinomycin (actinomycin D), Mitomycin C, Plycamycin (Mithramycin).

Various miscellaneous agents can also be used as therapeutic agents for the treatment of bladder or other disorders according to further embodiments of the invention. Exemplary miscellaneous agents include Cisplatin, Carboplatin, Asparaginase, hydroxyurea, Mitotane (o,p′-DDD; Lysodren), Anti-Estrogen (tamoxifen citrate), Corticosteroid (Prednisone); or Mebendazole (also referred to as Mebendozole).

The therapeutic solutions used to combat infections and their sequelae may include agents such as, but not limited to, antiseptic, antibacterial, antifungal, immunotherapeutic, immunosuppressive, chemotherapeutic, pH modifying, and other glycosaminoglycan (GAG) layer enhancing agents. The agent and the amount of the agent to be included in the solution are well within the determination of those skilled in the art.

Examples of antibacterial agents include, but are not limited to, aminoglycoside, cephalosporin, gentamycin, macrolide, nitrofurantoin, penicillin, quinolone, sulphonamide, tetracycline, trimethoprim, bacitracin, neomycin, chlorhexidine and mandelamine. Antifungal (antiyeast) agents include, but are not limited to, amphotericin B and fluconazole. The antibacterial agent could be selected from the group consisting of lincomycin, erythromycin, dirithromycin, clindamycin, clarithromycin, azithromycin, ticarcillin, piperacillin, meziocillin, carbenicillin indanyl, bacampicillin, ampicillin, amoxicillin, amoxicillin-clavulanic acid, ampicillin-sulbactam, benzylpenicillin, cloxacillin, dicloxacillin, methicillin, oxacillin, penicillin G, penicillin V, piperacillin plus tazobactam, ticarcillin plus clavulanic acid, amikacin, gentamicin, kanamycin, neomycin, netilmicin, streptomycin, tobramycin, tetracycline, oxytetracycline, minocycline, methacycline, doxycycline, and demedocycline. Further, an antibiotic that is not for systemic use due to toxicity could be used in this direct instillation method. Also viral and parasitic infections of the bladder could be effectively treated by direct instillation.

The invention may be used to treat recurrent bacterial infections by instillation of agents to replace or enhance the glycosaminoglycan (GAG) layer lining the lumen of the bladder. An example of this being sodium hyaluronate. Another such treatment would be instillation of antibiotics.

Immunotherapeutic agents include, but are not limited to, bacterial cell extracts, mycobacterial cell wall extracts, live and inactivated bacillus Calmette-Guerin (BCG), BCG extracts, cytokines, interferons, interleukins, prostaglandins, and immune stimulants of viral, chemical and molecular biological origin effective for treating disorders of the bladder and the associated cystitis. Immunosuppressive agents include, but not limited to, prostaglandins (PGE.sub.2) and corticosteroids. Chemotherapeutic agents include, but are not limited to, cisplatin, cyclophosphamide, doxorubicin (adriamycin), vincristine, mitomicin-C and thiotepa. pH modifying agents include, but are not limited to, sodium acid phosphate and sodium bicarbonate. Glycosaminoglycans (in addition to HA) include, but are not limited to, heparin, heparan sulfates, pentosanpolysulfate, dermatan sulfates, chondroitin sulfates and keratanosulfates.

The two primary functions of the bladder namely storage and voiding are opposite to each other. As such, drugs used to treat these functional disorders may also have opposite actions. Therefore for one condition an antagonist would be used whereas its agonist would be used for its opposite function. An example of this is overactive bladder and detrusor hypotonia. For the former an antimuscarinic agent would be used while for the latter, a muscarinic agonist would be used.

The invention may be used to treat urge incontinence due to detrusor hyperreflexia or overactive bladder by bladder instillation. A variety of therapeutic agents can be used for such treatments and include but are not limited to botulinum toxin and related compounds, muscarinic receptor antagonists such as atropine, propantheline, oxybutynin, tolterodine, tropspium, solifenacin or darifenacin; calcium channel inhibitors or mixed action drugs such as propiverine, dicylomine or flavoxate, muscarinic receptor agonists, spasmolytics, antidepressants, adrenoreceptor alpha antagonists, adrenoreceptor alpha agonists, adrenoreceptor beta antagonists, adrenoreceptor beta agonists, adrenoreceptor beta-3 agonists, cyclo-oxygenase inhibitors, vanilloids receptor agonists, vanilloids receptor antagonists, purinergic receptor antagonist, purinergic receptor agonist, tachykinin receptor agonists, tachykinin receptor antagonists, vasoactive peptide receptor agonist, vasoactive peptide receptor antagonist, opioid receptor agonists, opioid receptor antagonists, and compounds that enhance or inhibit or modulate nitric oxide synthesis. For example neurogenic urinary dysfunction can be treated with an effective dose of a homovanilloid compound, in particular a compound selected from the group RTX, TYX, 20-homovanillyl-mezerein or 20-homovanillyl-12-deoxyphorbol-13-phenylacetate.

The invention may be used to treat sensory hypersensitivity of the bladder such as painful bladder syndrome and interstitial cystitis. Examples of therapeutic agents include but are not limited to agents to replace or enhance the glycosaminoglycan (GAG) layer lining the lumen of the bladder. Examples of these include sodium hyaluronate, heparin, sodium pentosan polysulfate and chondroitin sulfate. Other treatments for interstitial cystitis can include but are not limited to instillation of histamine (H-1) receptor antagonist for example hydroxyzine, H-2 receptor antagonsists such as cimetidine, modulators of nitric oxide synthetase activity such as l-arginine, anti-inflammatory agents such as corticosteroids, cyclo-oxygenase inhititors and bioflavoids. Other agents that may be effective for interstitial cystitis include but are not limited to antibiotics, cytotoxic agents such as methotrexate, mast cell stabilizers and inhibitors of active agents released by mast cell granules (eg: leukotrienes and montelukasts) calcium channel blockers such as niphedipine, prostaglandin analogues such as misoprostol, immunosuppressive agents such as cyclosporine, analgesics, anesthetics such as lidocaine, resiniferatoxin, capsaicin; Bacillus Calmette-Guerin (BCG), dimethyl sulfoxide (DMSO), antimuscarinic agents, muscle relaxants, membrane stabilizers, doxorubicin, botulinum toxin and hypochlorous acid. Prostatic hypertrophy could also be treated by intravesical instillation of therapies that can and cannot be given systemically. Examples of this are alpha blockers, 5-alpha reductase inhibitors and neurotoxins such as botulinum toxin.

The use of lidocaine, alkalinized lidocaine or lidocaine followed by bicarbonate to act as an analgesic or to reduce bladder irritation and hence reduce the need to void is important but other compounds with similar action could be used as well or instead of lidocaine.

The reservoir could also be used to instill a formulation or preparation that would enhance the duration of action of the therapeutic agent or result in prolonged delivery of the drug. An example of this could be the use of nanotechnology creating micelles made in different ways to contain pharmacologically active ingredients or even lipid-based vehicles. Such vehicles could be a lysosome or antibody-coated liposomes that are useful for targeting specific receptors for drug, peptide, polypeptide, nucleic acid delivery. In particular aspect, the liposomes could be coated with antibodies against nerve growth factor (NGF) receptor and containing NGF antisense nucleic acids, which could be used as a treatment for neurogenic bladder dysfunction.

The reservoir could also contain regents that could enhance the uptake of therapeutic agents. Reagents which can be used to enhance bladder uptake in the bladder epithelium can be grouped as either single compounds or as mixed reagents (i.e., mixtures of compounds). Exemplary single compounds include non-ionic surfactants, alcohols, polymers and ionic surfactants. Exemplary surfactants include: Poloxamer 407 (Pluronic.®. 127); poloxamer 188 (Pluronic.®. F68); Polidocanol; n-dodecyl-.beta.-D-glucop-yranoside (which can also be classified as a sugar-based surfactant); n-dodecyl-.beta.-D-maltoside (which can also be classified as a sugar-based surfactant); Tween.®. 20; Triton.®. X-100; Forlan.®. C-24 (PEG Cholesterol); decyl-.beta.-D-maltoside (which can also be classified as a sugar-based surfactant); 6-cyclohexylhexyl-.beta.-D-malto-side (which can also be classified as a sugar-based surfactant); and sodium tetradecyl sulfate (e.g., Tromboject.®.). Exemplary alcohols that can be used to enhance uptake by the bladder epithelium according to the invention include benzyl alcohol and ethanol. Exemplary polymers that can be used to enhance uptake by the bladder epithelium according to the invention include HPMC 2910, PVA, and poly-lysine. Exemplary ionic surfactants that can be used to enhance uptake by the bladder epithelium according to the invention include: DC-Chol [Cholesteryl 3.beta.-N-(dimethylaminoethyl) carbamate]; the sodium salt of dodecyl benzenesulfonic acid; and sodium dodecyl sulfate. Exemplary mixed reagents that can be used to enhance uptake by the bladder epithelium according to the invention include: In vivo GeneSHUTTLE.™. (a reagent comprising DOTAP+Cholesterol available from Qbiogene of Carlsbad, Calif.) and oxychlorosene (i.e., sodium dodecylbenzenesulphonic acid/hypochlorous acid complex).

While the preferred embodiments of the invention have been shown and described, modifications thereof can be made by one skilled in the art without departing from the spirit and teachings of the invention. The embodiments described herein are exemplary only, and are not limiting. Many variations and modifications of the invention and apparatus disclosed herein are possible and are within the scope of the invention. All of the publications cited in the present application are hereby incorporated by reference herein.

What is claimed is:

1. A device for instilling a fluid into a bladder comprising:
a catheter member having a first end with a first aperture and a second end with a second aperture;
a channel connecting the first aperture with the second aperture;
valve means disposed within the channel to prevent fluid flow from said first opening toward said second opening;
a reservoir connected to the second aperture.

2. The device of claim 1 wherein said channel is of substantially uniform diameter from said first opening to said second opening.

3. The device of claim 1 wherein said reservoir is detachable.

4. The device of claim 1 wherein said reservoir is compressible.

5. The device of claim 1 further comprising a membrane separating the reservoir from the catheter which may be broken to allow the contents of the reservoir to flow into the bladder when pressure is applied to the membrane.

6. A device for emptying the bladder and instilling a fluid into the bladder comprising:
a catheter member having a first end with a first aperture and a second end with a second aperture and a third aperture;
a first channel within the catheter member connecting the first aperture with the second aperture;
a second channel within the catheter member, connecting the first aperture with the third aperture;
valve means connected to the second channel to control fluid flow through the second channel;
a reservoir connected to the second aperture.

7. The device of claim 6 wherein said reservoir is detachable.

8. The device of claim 6 wherein said reservoir is compressible.

9. The device of claim 6 further comprising a membrane separating the reservoir from the catheter which may be broken to allow the contents of the reservoir to flow into the bladder when pressure is applied to the membrane.

10. The device of claim 6 further comprising a valve means disposed within the first channel to prevent flow from the first aperture to the second aperture.

11. A method for treating a bladder disease in a patient, comprising self-administering by trans-urethral catheterization into the bladder by said patient of an effective amount of a therapeutic compound for the treatment of the bladder.

12. The method of claim 11, wherein the bladder disease is selected from the group consisting essentially of interstitial cystitis, painful bladder syndrome, urge incontinence, neurogenic incontinence, unstable bladder, detrusor overactivity, overactive bladder, frequency urgency syndrome, malignant disease of the bladder, and intractable bladder infection.

13. The method of claim 11, wherein the therapeutic compound is selected from the group consisting essentially of hyaluronic acid, heparin, chondroitin, pentsanpolysulphate (PPS), lidocaine, resiniferatoxin, capsaicin, Bacillus Calmette-Guerin (BCG), dimethyl sulfoxide (DMSO), an antimuscarinic agent, neurotoxin such as botulinum toxin, doxorubicin or hypochlorous acid, an antimuscarinic agent or its derivative, atropine, propantheline, oxybutynin, tolterodine, tropspium, solifenacin, darifenacin, calcium channel inhibitors, propiverine, dicylomine, flavoxate, beta-adrenoreceptor agonist, cyclo-oxygenase (COX) inhibitors types I or II, aspirin, indomethicin, ketorolac, etodolac, meloxicam, ibuprofen, flurbiprofen, naproxen, ketoprofen, diclofenac, nabumetone, sulfasalazine, oxaprzin, celecoxib, baclofen, capsaicin, resiniferatoxin, neurotoxins, botulinum toxin, inhibitor of the vanilloid or purinergic receptors, modulator of nitric oxide metabolism, sex hormone, estrogen, anticancer drugs, vincristine, doxorubicin, mitoxantrone, camptothecin, cisplatin, bleomycin, cyclophosphamide, methotrexate, streptozotocin, actinomycin D, vincristine, vinblastine, cystine arabinoside, anthracyclines, alkylative agents, platinum compounds, antimetabolites, nucleoside analogs, methotrexate, purine and pyrimidine analogs, adriamycin, daunomycin, mitomycin, epirubicin, 5-FU, and aclacinomycin.

14. The method of claim 13, further comprising self-administration of an antimicrobial, antibiotic, or antiviral agent.

15. The method of claim 13, further comprising self-administration of an analgesic agent.

16. The method of claim 11, wherein the therapeutic compound is self-administered at a time which will maximize the time the therapeutic compound remains in the bladder prior to voiding.

17. The method of claim 16, wherein the therapeutic compound is self-administered after the patient voids the bladder and before the patient goes to sleep.
